(19) United States
(12) Patent Application Publication
(10) Pub. No.: US 2006/0094894 A1
(43) Pub. Date: May 4, 2006

(54) Acylated esters

(76) Inventor: James F. Day, Winston-Salem, NC (US)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/978,584

(22) Filed: Nov. 2, 2004

(51) Int. Cl. C07C 69/66

(52) U.S. Cl. 560/180

(57) ABSTRACT

Acylated citrate esters are made with a polishing step that includes contact with a polishing composition having a fatty acid absorbent to remove residual odor-forming precursors from the resulting acylated ester.

### FIELD OF THE INVENTION

The invention relates to a method for making citrate esters that have reduced or eliminated odors when formulated into plastic products.

### BACKGROUND OF THE INVENTION

In medical containers that contact blood products, such a blood bags, certain plasticizers appear able to suppress hemolysis of red blood cells stored therein. Citrate esters having the formula shown in U.S. Pat. No. 5,026,347 which include acetyltri-n-hexyl citrate, acetyltri-n-(hexyl/octyl/decyl) citrate, acetyltri-n-(octyl/decyl) citrate, and n-butyryltri-n-hexyl citrate are particularly useful.

Citrate esters are recognized as useful plasticizers for polyvinyl chloride (see, U.S. Pat. Nos. 4,710,532; 4,789,700; 4,711,922; 4,824,893; 4,870,204; 4,883,905; 4,892,967; 4,931,583; 4,954,649; and 5,055,609), ultrahigh molecular weight PVC (see, U.S. Pat. Nos. 5,721,024; 5,968,619; and 6,060,138), polyolefin copolymers (see, U.S. Pat. Nos. 5,026,347; 5,100,401; and 5,167,657), and vinylidene chloride (see, U.S. Pat. Nos. 5,726,229; 6,133,352). The disclosures of each of the patents cited herein and hereafter are expressly incorporated by reference.

The manufacturing processes for acylated citrate esters generally include (a) an esterification step using a catalyzed reaction of an alcohol and an acid, (b) neutralization and steam stripping of excess alcohol, (c) acylation using a suitable acid or acid anhydride with an acid acylation catalyst, (d) and recovery steps that include neutralization, bleaching, and separation. See, Published US Patent Application No. 2002/011508 and U.S. Pat. No. 6,355,817 (two doses of esterification catalyst); U.S. Pat. No. 6,403,825 (continuous process for making citrate ester by continuous phase separation); and U.S. Pat. No. 4,710,352.

Unfortunately, the resulting acylated citrate ester product from these conventional processes and from other acylation processes using other carbonyl-providing component and exhibits an objectionable, although mild, odor when formulated, formed into a container, and subjected to elevated temperatures that approximate sterilizing conditions. See, U.S. Pat. No. 4,710,352 in the table at column 6 (“mild” odor at 25° C. after heating at 150° C. for two hours). It would be desirable to have a citrate ester plasticizer that was free or at least essentially free of detectable odors at 25° C. after heating at 150° C. for two hours.

### SUMMARY OF THE INVENTION

It is an objective of the invention to provide an acylated ester and process for making such esters that is effective at removing odor-forming residual components from the citrate ester product.

It is a further object of the invention to provide a flexible plastic stock material and products made therefrom that include a polymer and a plasticizer containing the acylated citrate ester that is free or essentially free of fatty acid odors that are perceptible to humans at 25° C. after being heated at 150° C. for two hours.

In accordance with these and other objectives of the invention that will become apparent from the description herein, a process for making an acylated ester according to the invention comprises:
(a) esterifying an alcohol or alcohol mixture with a carbonyl-contributing compound in the presence of an esterification catalyst under esterification conditions and removing excess alcohol to form an ester-containing reaction mixture,
(b) acylating the ester-containing reaction mixture with an acid or acid anhydride under acylation conditions with an acylation catalyst for a time sufficient to form an acylated ester,
(c) removing by-product fatty acids and neutralizing the acylated ester, and
(d) polishing the acylated ester with a polishing composition comprising an absorbent for fatty acids.

Acylated esters made according to the invention exhibit no, or essentially no, odor that is perceptible to normally healthy humans at 25° C. after being heated at 150° C. for two hours. When used as plasticizers, they exhibit good heat stability that make them well-suited for use in medical equipment (blood bags, tubing, containers, etc.) that must be sterilized before use. The absence of residual odors for medical products using a plasticizer made with an acylated ester according to the invention avoid the formation of objectionable odors in medical facilities for the comfort of the personnel and for avoiding the formation of obscuring odors in a medically sterile environment where the presence of an odor can be an indicator of infection or equipment malfunction.

### DETAILED DESCRIPTION OF THE INVENTION

Acylated esters are very useful as plasticizers for polymeric products, but nobody wants to endure objectionable odors when using the products. This is particularly true in medical environments where there is an ever-present threat of infection and a continuing standard of absolute cleanliness. In such situations, objectionable odors from sterilized plastic products are overtly prominent, objectionable, and may hinder the continuing efforts to detect odors that represent warning signs of possible infection or equipment malfunction.

The Manufacturing Process

The present invention provides a manufacturing process for making acylated esters, particularly those that can be used as plasticizers for polymeric products and the resulting plasticized polymeric product that incorporates these plasticizers. This manufacturing process includes the esterification of an alcohol with a carbonyl-contributing compound, e.g., an acid or acid precursor, and acylating the ester-containing intermediate with an acid or acid anhydride to make an acylated ester. This acylated ester is then separated and polished with a polishing composition that includes an absorbent for fatty acids. Preferably, a bleaching compound is used with the polishing composition. It is believed that residual levels of fatty acids or fatty acid precursors from the esterification-acylation processes are the source of the residual odors that are perceptible following exposure to elevated temperatures and ambient oxygen levels. Treatment with the polishing composition removes these odor-forming precursors from the acylated ester product before they can degrade and produce objectionable volatile, objectionable organic odors. The result is an acylated ester that has no more, and preferably less, than a de minimis odor after heating to 150° C. for two hours and cooling to 25° C. Indeed, any odor is far less than a mild odor (see, U.S. Pat. No. 4,710,532 and progeny) and would best be subjectively characterized as no more than “barely perceptible”, preferably, “essentially undetectable”, and even more preferably “odor-free” to a normally healthy human.

The acylated esters of the invention exhibit a quantifiably surprising heat stability either in the acylated ester form (neat) or when formulated in a plasticizing amount with a polymer when compared to the existing commercial state of the art. For example, n-butyryl-tri-n-hexyl citrate exhibits less than 10% of volatile butyric acid over the neat sample when subjected to a heat exposure test that includes heating to 150° C. for two hours, cooled to 25° C., and tested by GC-MS. When formulated with medium weight PVC, similarly low results were obtained. Exemplary heat stability for the invention is seen by a substantially pure citrate ester in neat form that exhibits less than 2 ppm, preferably less than 1.5 ppm volatile fatty acids following the heat exposure test. When formulated as a plasticizer with a medium weight PVC, the plasticized formulation desirably exhibits less than 1 ppm, preferably less than 0.5, and especially preferred is less than 0.25 ppm volatile fatty acids following the heat exposure test. The fatty acids are likely the degradation products of the acylation compound or compounds used to make the acylated ester.

As the alcohol component in the esterification step of the invention process, suitable are most primary and secondary C₁-C₃₀ monohydric or polyhydric, substituted or unsubstituted alkanols and alkenols, such as, methanol, ethanol, chloroethanol, cyanoethanol, ethoxyethanol, phenylethanol, n-propanol, 2-chloropropanol-1,3-bromo-propanol-1,2,2-dichloropropanol-1, isopropanol, propanol-2,2-nitrobutanol-1,2-nitrobutanol-1,2-methylpentanol-1,2-methyl pentanol-3, the primary and secondary octanols, n-dodecanol, 6-dodecnol, lauryl, myristyl, stearyl, 2-propenol-1,2-butenol-1,3-pentenol-1, ethylene glycol, trimethylene glycol, tetramethylene glycol, pentamethylene glycol, glycerol, 1,4-butanediol, decane-1,10-diol, pentadecane-1,15-diol, pentacosane-1,25-diol, 2,4-hexadiene-1,6-diol, 2,4-octadiene-1,8-diol, and aromatic alcohols such as benzyl alcohol, o-, m- and p-methoxy alcohol, o-, m- and p-nitrobenzyl alcohol, o-, m- and p-methyl benzyl alcohol, phenyl ethyl alcohol, triphenyl ethyl alcohol, o-, m- and p-benzyl benzyl alcohol, alpha-naphthyl-ethyl alcohol, beta-naphthyl ethyl alcohol, naphthylene-1,2-diethyl alcohol, phenylene-1,3,5-triethyl alcohol, phenylene-1,4-dioctyl alcohols, and mixtures thereof. This includes higher Guerbet alcohols which are beta carbon branched dimer alcohols having ten to twenty-six carbon atoms.

Polyhydroxy compounds are represented by the general formula:
R(OH)ₙ
wherein:

R is an alkyl, alkenyl, or aralkyl hydrocarbyl group, and

n is at least 2.

The hydrocarbyl group R may contain from about 2 to 20 or more carbon atoms, and the hydrocarbyl group may also contain substituents such as chlorine, nitrogen, and/or oxygen atoms. The polyhydroxy compounds generally will contain from about 2 to 10 hydroxy groups and more preferably from about 2 to 6 hydroxy groups. The polyhydroxy compound may contain one or more oxyalkylene groups and, thus, the polyhydroxy compounds include compounds such as polyetherpolyols. The number of carbon atoms and number of hydroxy groups contained in the polyhydroxy compound used to form the carboxylic esters may vary over a wide range.

The following alcohols are particularly useful as polyhydroxyls: neopentyl glycol, 2,2-dimethylol butane, trimethylol ethane, trimethylol propane, trimethylol butane, mono and technical grade (i.e., 88% mono, 10% di and 1-2% tri) pentaerythritol, dipentaerythritol, ethylene glycol, propylene glycol and polyalkylene glycols (e.g., polyethylene glycols, polypropylene glycols, polybutylene glycols, etc., and blends thereof such as a polymerized mixture of ethylene glycol and propylene glycol).

Preferred alcohols include linear as well as branched alcohols of any source. Preferred alcohols generally have four to 14 carbon atoms in the molecule and may be used as a reactant as a single type of alcohol (e.g., butanol or hexanol in a technical grade of purity) or may be used as mixtures of isomeric forms of alcohols of the same number of carbon atoms or alcohols of different numbers of carbon atoms (e.g., mixtures containing hexanol, octanol, and decanol or just octanol or decanol). The proportions of the specific alcohols used in the esterification reactant mixture can be varied to adjust the properties of the resulting product.

The carbonyl-contributing compound for the esterification reaction include carboxylic acids. Carboxylic acids which undergo esterification (i.e., mono or poly-basic acids, preferably dibasic or tribasic acids) can be aliphatic, cyclo-aliphatic or aromatic, they can be substituted or unsubstituted, saturated or unsaturated, or they can be blends of acids. Representative acids include acetic, hydroxyacetic, chloroacetic, bromoacetic, cyanoacetic, 5-phenylacetic, triphenyl acetic, propionic, halopropionic, lactic, beta-hydroxy propionic, n-butyric, isobutyric, n-valeric, isovaleric, 5-phenyl-n-valeric, n-heptanoic, caproic, pelargonic, caprylic, lauric, palmitic, lignoceric, alpha-hydroxy lignoceric, malonic, succinic, glutaric, adipic, pimelic, azelaic, sebacic, decane-1,10-dicarboxylic, pentadecane-1,15-dicarboxylic, pentacosane-1,25-dicarboxylic, 1,2,3-propane tricarboxylic, citric, acrylic, alpha-chloro acrylic, beta-chloro acrylic, beta-bromo acrylic, beta-phenyl acrylic, methacrylic, vinyl acetic, crotonic, angelic, tiglic, undecylenic, oleic, erucic, linoleic, linolenic, maleic, fumaric, mesaconic, citraconic, itaconic, mucconic, aconitic, myristic, stearic, isostearic, branched C₅ and C₁₀ (e.g., 3,5,5-trimethylhexanoic), branched C₁₇, C₁₉, C₂₁, etc., acids, and mixtures thereof. As described in U.S. Pat. No. 6,403,825 fermentation broths that contain citric acids may be used as a source of carbonyl-contributing compounds.

Among the alicyclic acids are cyclopropane carboxylic, cyclobutane carboxylic, cyclopentane carboxylic, cycloheptane carboxylic, cyclohexane carboxylic, 2-hydroxy cyclohexane carboxylic, 1,1-cyclopropane dicarboxylic, 1,2-cyclobutane dicarboxylic, 1,3-cyclobutane dicarboxylic, 1,4-cyclohexane dicarboxylic, cyclohexane-1,2,3,4,5,6-hexacarboxylic, cyclopentene-2-carboxylic, 1-cyclohexene-1-carboxylic, hydrocapric, cyclohexadiene-1,2-dicarboxylic, 1,3-cyclohexadiene-1,4-dicarboxylic, and mixtures thereof.

The aromatic acids include benzoic, o-, m- and p-chloro and bromo benzoic, o-, m- and p-hydroxy benzoic, o-, m- and p-nitrobenzoic, o-, m- and p-methoxy benzoic, alpha-napthoic, beta-naphthoic, o-, m- and p-methyl benzoic, o-, m- and p-ethyl benzoic, p-phenyl benzoic, phthalic (1,2-benzene-dicarboxylate), isophthalic, terephthalic, hydroxy phthalic, 2,3-dimethyl benzoic, benzene-1,2,4-tricarboxylic, benzene-1,3,5-tricarboxylic, benzene-1,2,4,5-tetracarboxylic, diacids of naphthalenes and trimellitic, and mixtures thereof. An especially preferred carbonyl-contributing compound is citric acid.

When polyols are used to form an ester the following acids are preferred: neopentanoic acid, neoheptanoic, neo-octanoic acid, neononanoic acid, neodecanoic acid, 2-ethyl hexanoic acid, oxo-heptanoic acid (i.e., a mix of isomers derived from oxonation/oxidation of hexenes), oxo-decanoic acid (i.e., a mix of isomers derived from oxonation or oxidation of mixed nonenes), oxo-octanoic acid (i.e., a mix of isomers derived from oxonation or oxidation of mixed heptenes), 3,5,5-trimethylhexanoic acid, linear C₅-C₁₈ alkanoic acids, and mixtures thereof.

The esterification process according to the present invention is conducted in the presence of an esterification catalyst in an amount sufficient to promote and accelerate the esterification reaction. Esterification catalysts include organometallic catalysts and acid catalysts.

Typical organometallic esterification catalysts include titanium, zirconium and tin catalysts such as titanium, zirconium and tin alkoxides, carboxylates and chelates. Exemplary titanium alkoxides which can be used as catalysts include tetramethyl titanates, tetraethyl titanates, tetrapropyl titanates, tetra-isopropyl titanates, tetrabutyl titanates, tetrapentyl titanates, tetrahexyl titanates, tetra-octyl titanates, tetranonyl titanates, tetradodecyl titanates, tetrahexadecyl titanates, tetra-octadecyl titanates, tetradecyl titanates, tetraheptyl titanates, tetraphenyl titanates, and mixtures thereof. The alkoxy groups on the titanium atom can all be the same or they can be different. The tin or zirconium counterparts of the above alcoholates can be substituted in whole or in part as catalysts.

The titanium carboxylates, which serve as esterification catalysts, are polymeric materials having at least one acyl group for each titanium atom. Typical titanium acylates which can be employed as catalysts include acylates from 2 to 18 carbon atoms, such as hydroxy titanium acetate, hydroxyl titanium butyrate, hydroxy titanium pentanoate, hydroxy titanium hexanoate, hydroxy titanium octanoate, hydroxy titanium decanoate, hydroxy titanium dodecanoate, hydroxy titanium tetradecanoate, hydroxy titanium hexadecanoate, hydroxy titanium octadecanoate, hydroxy titanium oleate, hydroxy titanium soya acylate, hydroxy titanium linseed acylate, hydroxy titanium castor acylate, hydroxy titanium tall oil acylate, hydroxy titanium coconut acylate, methoxy titanium acetate, ethoxy titanium butyrate, isopropoxy titanium pentanoate, butoxy titanium hexanoate, isopropoxy titanium octanoate, isopropoxy titanium decanoate, isopropyl titanium dodecanoate, isopropoxy titanium tetradecanoate, isopropoxy hexadecanoate, isopropoxy octadecanoate, isopropoxy titanium oleate, isopropoxy titanium soya acylate, isopropoxy linseed acylate, isopropoxy coconut acylate and mixtures thereof. The alkoxy group of the acylate can vary from 1 to 20 carbon atoms. The corresponding tin and zirconium carboxylates can be substituted, in whole or in part, as catalysts.

Titanium chelates are formed by reacting a titanium compound with a polyfunctional molecule including polyols such as glycols or glycerine and amino alcohols, amino acids, hydroxy acids, and polycarboxylic acids. Typical chelated esters which serve as catalysts include tetra-ethylene glycol titanate, tetrapropylene glycol titanate, tetrabutylene glycol titanate, tetra-octylene glycol titanate and tetrapolyethylene glycol titanate, dibutoxy di-(ethylene glycol) titanate, di-isopropoxy di-(octylene glycol) titanates, dimethoxy di-(octylene glycol) titanates, diethyoxy di-(octylene glycol) titanates, tetratriethanol amine titanate, tetratriethanol amine-N-oleate titanate, triethanol amine-N-stearate titanate, triethanol amine-N-linseed acid salt titanate, dibutoxy titanate, dipropoxy titanate, dimethoxy titanate, diethoxy titanate, other dialkoxy dipropoxy, dimethoxy, diethoxy titanates, other dialkoxy di-(amino alcohol) titanates and mixtures thereof. The corresponding tin and zirconium chelates can be useful as catalysts.

Suitable acid esterification catalysts include methanesulfonic acid, sulfuric acid, formic acid, polyphosphoric acid, or p-toluene sulfonic acid which are dissolved in the reaction mixture or suspended as the pure compound.

Especially preferred esterification catalysts for use in the present invention include titanium, zirconium, or tin organometallic catalysts as well as sulfuric acid, methanesulfonic acid, p-toluene sulfonic acid. Of these, sulfuric acid, methanesulfonic acid, and tetra-n-butyl titanate are particularly preferred esterification catalysts.

Esterification catalyst can be added in one or more doses. See, U.S. Pat. No. 6,355,817. Total amounts of esterification catalyst will generally fall within a range from about 0.01 wt % to about 5 wt % based on the weight of the limiting reactant of the esterification reaction. The catalyst concentration is usually within the range from about 0.01 to 1.0, preferably 0.01 to 0.5 percent by weight relative to the limiting reactant in the esterification reaction. Which reactant is limiting is readily determinable by one skilled in this art using no more than the existing level of skill in the art.

Esterification conditions generally include a temperature within the range from about 110° to 140° C. has been found to be useful. Lower temperatures are not excluded if a slower reaction rate is desired or only partial reaction is intended. Increased or decreased pressure within the esterification reactor can be used, if desired.

The pressure in the reaction vessel(s) for the esterification reaction should be maintained at a level sufficient to reflux the alcohol or acid (acting as an entrainer for water) and the water while forming an ester from the reactants. Pressures ranging from a vacuum to low positive gauge pressure are generally used to carry out esterification reactions during the reaction cycle. The pressure of the reaction vessel is usually adjusted continually to ensure continuous vaporization and removal of water in the vapor. Typically, the initial reaction pressure is close to atmospheric pressure, for example 1 to 2 atm (101.3 to 202.6 kPa), and moves toward an increasing vacuum as the reaction proceeds. Preferably, the final reaction pressure ranges from 2 atm (202.6 kPa) to 100 mm Hg absolute (13.3 kPa). More preferably, the final reaction pressure ranges from 1.0 atm (101.3 kPa) to 200 mm Hg absolute (26.7 kPa). Most preferably, the final reaction pressure e ranges from 250 mm Hg absolute (33.3 kPa) to 350 mm Hg absolute (46.7 kPa).

Dewatering is preferably effected by azeotropic distillation during the esterification reaction with an entraining agent. Preferred entraining agents include C4-C10 alcohols in linear and cyclic conformations with cyclohexane, cyclopentane, and cyclohexane being especially preferred. See generally, published US Patent Application No. 2002/0111508 which is herein incorporated by reference. Sufficient quantities of such entraining agents are desirably used to maintain the esterification reaction sufficiently low, e.g., below 110° C., to promote the desired product.

The esterification step of the process according to the invention may also be accomplished in a batch process or using a continuous process. In the continuous process, two or more (e.g., 2-5) reaction vessels or reaction zones are employed. Each reactor vessel or zone contains a separate catalyst-to-feed contacting system and may include continuous removal of water to drive the reactions toward completion. The continuous process may allow a target conversion to be achieved at a higher space velocity, thus resulting in higher total process feed rates and economics.

Preferred esters for use as intermediates in the manufacture of plasticizers for flexible polymeric medical products are generally triesters made with linear alkyl alcohols having six, eight, or ten carbon atoms, or mixtures of such alcohols.

After removing reaction water, the ester-containing intermediate is further reacted under acylation conditions with an acid or acid anhydride and an acylation catalyst to make an acylated ester. Suitable acylation conditions include a temperature within the range from about 50° to about 110° C., preferably within the range from about 60° to about 80° C.

Useful acylating agents generally include anhydrides of mono- and poly-basic acids whose presence in even low amounts in the resulting acylated ester product can hydrolyze under the elevated temperatures and ambient oxygen levels of typically sterilization processes, such as those routinely encountered in the processing of products used in medical establishments. Such anhydrides include acetic anhydride, propionic anhydride, n-butyric anhydride, succinic anhydride, glutaric anhydride, adipic anhydride, pimellic anhydride, maleic anhydride, mesaconic anhydride, citraconic anhydride, glutaconic anhydride, itaconic anhydride, phthalic anhydride, benzoic anhydride, nadic anhydride, methyl nadic anhydride, hexahydrophthalic anhydride, trimellitic anhydride and mixed anhydrides of monobasic acids. Another anhydride is pyromellitic dianhydride. Especially preferred is an acylating agent that includes or consists essentially of acetic anhydride or n-butyric anhydride with the associated formation of acetyltri-n-hexyl citrate or n-butyryltri-n-hexyl citrate plasticizer. These citrates are well-suited for use in medical containers of flexible polymers with n-butyryltri-n-hexyl citrate being known to suppress hemolysis of red blood stored in flexible polymeric bags that use this citrate as a plasticizer. See, U.S. Pat. No. 5,026,347 whose disclosure is incorporated herein by reference.

Acid catalysts and organotitanates are generally used to promote the acylation reactions. A preferred acid catalyst is concentrated sulfuric acid. A preferred organotitanate is tetra-n-butyl titanate. See generally, U.S. Pat. Nos. 4,710,532 and 4,711,922 which are herein incorporated by reference.

Following the formation reaction, the acylated ester product is neutralized and bleached. The neutralization step is used to remove unreacted acids and anhydrides as well as any acid acylation catalyst that was used. Typically useful neutralization agents are alkaline in nature, e.g., sodium carbonate or sodium hydroxide in aqueous solution. Preferably, treatment first with a Group II alkaline earth metal salt (first Group II alkaline earth metal salt) is used instead of a water wash to remove acidity. A weak base is preferred, such as magnesium acetate, to neutralize the acid. Alternatively, calcium acetate may be used. Use of an acetate ensures that the pH will be less than 7.0 which is critical because the titanium catalyst is sensitive to, and is destroyed by, alkaline pH. The first Group II alkaline earth metal salt is used at 0.01 to 10 percent of the weight of the reaction mass (defined as the weight of the original reactants, preferably at 0.1 to 0.5 weight percent, most preferably at stoichiometric levels equal to that of the residual acid.

If hot water (about 90° C.) is used, typically 3-4, 300-ml aliquots of water (usually about 1 part water to 2 parts acylated ester) have been found to be sufficient to remove the residual acid in the aqueous phase to less than 0.1%, when a mole of anhydride is using in the starting reaction mix. If less water is used, more washes are generally required to achieve the results of the invention. The water wash treatment, while being effective, is substantially more time-consuming than adding a Group II alkaline earth metal salt, which is the preferred treatment.

The neutralized acylated ester product is then bleached by contact with a dilute aqueous solution containing an oxidizing compound and an absorbent for residual fatty acids. Useful oxidizing agents are relatively weak and water soluble to facilitate separation from the organic acylated ester product. Suitable oxidizing agents include hydrogen peroxide and similar bleaching compounds. Preferred is a dilute solution containing 0.05-1 wt % hydrogen peroxide and especially a bleaching solution that contains 0.15-0.35 wt % hydrogen peroxide.

Simultaneously with the bleaching step or in a separate treatment is a polishing of the acylated ester product with a polishing composition that comprises a fatty acid absorbent. Fatty acid absorbents useful in the invention can be any material that does not adversely affect the polymer of the plasticized product and which is capable of absorbing or neutralizing low, residual levels of fatty acids in solution. For example, butyric acid has a noxious odor like rancid butter whose presence, even in residual levels, may be responsible for objectionable odors following sterilization at elevated temperatures and uncontrolled ambient oxygen concentrations. It is believed that the fatty acid absorbent in the polishing composition helps to remove residual alkyl acid anhydride that remains from the acylation step or, if the absorbent remains in the polished product at residue levels, which is formed during exposure to high sterilization heats (e.g., about 150° C.) encountered with medical products. The use of the fatty acid absorbent in the polishing step results in an odor-free, or effectively odor-free acylated ester that is free of objectionable odors immediately after production and which remains odor-free following exposure to sterilization temperatures and conditions (e.g., a temperature of 150° C. for two hours followed by cooling to 25° C.) as either the neat acylated ester or when formulated as a plasticizer with a polymer.

A suitable bench test of the acylated ester sample can be performed by warming a sample to about 110° C. in a closed container, dipping a test strip of a neutral absorbent material into the warmed sample, and smelling the test strip. Preferably, this “sniff” test is performed in an area that is free of obscuring or distracting odors, quiescent air, and by a human in good health with at least an average ability to detect odors. Use of an expert in odor detection, like a perfume chemist, would provide an even higher degree of sensitivity to discernible odors. Such a bench “sniff” test would allow a faster test of odor residues without having to expose the sample to sterilization temperatures followed by cooling to room temperatures before testing for odors.

Use of sterilization conditions and cooling may still be desirable in testing formulated polymeric products for heat stability. Such a test is seen by exposure to elevated heats and duration of the neat acylated ester or formulated resin containing the present plasticizer in a sufficient amount to render the resinous material flexible. A suitable would be 150° C. for two hours followed by cooling to 25° C. and analysis with a GC-MS at a 10× dilution rate. The BTHC neat product of the present invention will exhibit a volatile butyric acid concentration of less than 2.5 ppm (allowing for the 10× dilution) as an indication both of its essentially odor-free nature and its enhanced stability relative to BTHC products that have been made without use of a polishing step for absorption of residual odor-forming precursor compounds.

Preferred fatty acid absorbents for use in the polishing process according to the invention are separable from the bleached, acylated ester and do not adversely affect performance of the ester. Most preferable are fatty acid absorbents that are solids that are insoluble in the acylated ester solution and which may be readily recovered for reuse or disposal, as needed.

Examples of suitable fatty acid absorbents may or may not be the same as the earlier used neutralizing agent and would include alkali and alkaline earth metal silicates, oxides, hydroxides, cyclodextrins, clays, crystalline or amorphous aluminosilicates either with or without surface modification by an alkylamine (see, U.S. Pat. No. 6,627,084). Preferred fatty acid absorbents to be used in the polishing composition include lithium silicate, calcium silicate, magnesium silicate, calcium oxide, magnesium oxide, lithium hydroxide, calcium hydroxide, magnesium hydroxide, beta-cyclodextrin, alpha-cyclodextrin, gamma-cyclodextrin, derivatives of said cyclodextrins, and mixtures thereof or from the group consisting of methyl substituted cyclodextrins, ethyl substituted cyclodextrins, hydroxyalkyl substituted cyclodextrins, branched cyclodextrins, cationic cyclodextrins, quaternary ammonium cyclodextrins, anionic cyclodextrins, amphoteric cyclodextrins, cyclodextrins wherein at least one glucopyranose unit has a 3-6-anhydro-cyclomalto structure, and mixtures thereof.

The amount of fatty acid absorbent used in the polishing solution should be sufficient to absorb up to 1 wt % fatty acid residues in the acylated ester product and produce a polished product. While the specific amount of absorbent used will depend on the absorbency of the material, it is generally sufficient to use 0.15-0.5 grams of magnesium silicate per 100 ml of acylated ester product. The bleaching and polishing conditions are generally performed at a temperature within the range from about 50° C. to about 95° C., preferably about 90° C. for 1 to 4 hours followed by vacuum distillation at 90°-140° C. to remove residual water.

The acylated ester product is preferably an acylated triester made by the esterification of citric acid with one or more C6-C10 alcohols to make a citrate triester and the subsequent acylation of the citrate triester with a C2-C10 acid anhydride to make an acylated triester citrate that has less than 100 ppm, preferably less than 50 ppm, and most preferably 0-25 ppm of the acid anhydride following the polishing process.

Plasticized Compositions

The acylated ester product of the invention can be used as a plasticizer for a wide variety of flexible plastic polymers. Examples of suitable polymers that can be formulated with the plasticizer of the present invention include polyvinyl chloride (PVC) (see, U.S. Pat. No. 4,711,922), ultrahigh molecular weight PVC (see, U.S. Pat. No. 5,968,619), non-PVC polymers such as polypropylene and a polyolefin copolymer containing a central block of ethylene and butylenes units with terminal blocks of polystyrene (as described in U.S. Pat. No. 5,026,347), vinylidene chloride (see, U.S. Pat. No. 6,133,532).

The acylated ester product of the invention is well suited for use as a medically acceptable plasticizer and is characterized by the absence of a perceptible plasticizer residue or by-product odor to humans following sterilization heating at 150° C. for two hours and cooling to 25° C. without the use of external odor masking agents or disinfectants that have been added for the purpose of suppressing or masking residual plasticizer odors in the final, sterilized product. It is important that the odor measurement be made after the product has been exposed to the high heats of sterilization for an extended period of time. Such conditions more accurately reflect the true heat history of the products that will be used, and the high heat in the presence of ambient oxygen and water vapor will cause any residual acid anhydride to hydrolyze to the corresponding volatile acid with the corresponding noxious odors.

The following table 1 includes additional properties that characterize the plasticized plastic compositions of the invention:

TABLE 1

| Property | Typical | Preferred |
|---|---|---|
| Purity (wt %) | ≧99 | ≧99 |
| Color APHA | ≦60 | ≦50 |
| Neutralization No. (mg KOH/g) | ≦0.25 | ≦0.20 |
| Specific Gravity (25° C.) | 0.98-1.1 | 0.99-1.01 |
| Viscosity (25° C., cps) | 20-40 | 25-35 |
| Odor (at 25° C.) | None* | None* |

After Heating at 150° C. for 2 hours:

| | | |
|---|---|---|
| Color APHA | ≦60 | ≦50 |
| Neutralization No. (mg KOH/g) | ≦0.25 | ≦0.20 |
| Odor (at 25° C.) | De minimis, | None* |
| | essentially none, | |
| | or none* | |

*Effectively no perceptible odor to a normally healthy human at a distance of 1 meter in a closed room. Preferably, there is essentially no perceptible odor to a human experienced in the detection of perfumes and similar subtle odors.

Flexible plastics that will use the plasticizers of the present invention will generally contain 40-90 wt % of the polymer or polymeric components and 10-60 wt % of the plasticizer. Higher or lower amounts of the plasticizing acylated ester and polymeric components may be used depending on the specific properties of the polymer and provided that the mechanical properties of the polymeric container are not adversely affected.

A wide variety of flexible polymer products can be made using the odor-free acylated ester plasticizer of the invention. Suitable examples include blood bags, flexible tubing, gloves, films of one or more layers that may be used to make medically acceptable product containers, packaging used in indirect food contact like food wraps and children toys (especially those with a high likelihood of being used in or in contact with mouth tissues. The present acylated ester product can also be used in other conventional PVC, polyvinylidene chloride (PVDC), polyvinylidene fluoride (PVDF), or similar halogenated resin applications, e.g., PVC back coating, automobile interiors to prevent fogging from the release and condensation of plasticizer following exposure to high temperatures, wire and cable insulation, resilient flooring, and the like.

### EXAMPLES

### Example 1

To a 1 liter, four neck flask equipped with stirrer, immersion thermometer, sub-surface nitrogen sparge tube and a barrett trap with condenser was charged 330 grams n-hexanol, 180 grams citric acid and 50 ml heptane. The reaction mass was heated with a heating mantle to reflux (approx. 120° C.), and 1.5 grams of tetra-n-butyl titanate esterification catalyst was added. The reaction mass was esterified at 120-140° C. until the acidity was less than 2 mg KOH/g acidity. The pressure was lowered using a vacuum pump to 20 mm Hg thereby distilling off the excess n-hexanol and heptane azeotrope. The ester-containing reaction mass was then steam-stripped with live steam at a temperature of 130-140° C. and full vacuum until no residual n-hexanol was detectable by gas chromatography analysis.

The ester-containing reaction mass is cooled to 90° C., and 0.4 grams concentrated sulfuric acid is added dropwise with stirring. N-butyric anhydride (166 grams) is added dropwise with stirring at a rate to maintain an acylation temperature of between 90-110° C. The acylation reaction mixture is stirred and allowed to proceed for 2 hours to make an acylated ester reaction mass. By-product butyric acid is removed with vacuum distillation at 110° C.

The acylated ester reaction mass is cooled to 70° C. and neutralized to a pH of 10 with a sufficient quantity of a neutralizing solution containing 10% aqueous sodium carbonate. The aqueous layer is decanted, and the acylated ester-containing organic product layer is washed once with 250 ml water. A bleaching and polishing solution containing 4 grams of 30% hydrogen peroxide and 4 gram of magnesium silicate is added dropwise at 70° C. The bleaching/polishing steps are allowed to continue for 30 minutes at 70° C. The bleached and polished acylated ester mass is then dehydrated to less than 0.1% water content by Karl Fischer titration using full vacuum. This acylated ester mass is filtered to remove magnesium silicate. The amount of 488 grams of colorless (20 APHA) and odorless product with an assay of 99.8% is obtained.

### Example 2

Tetra-n-butyl titanate esterification catalyst is replaced by 2.5 grams of tetra-n-butyl zirconate. The yield and product quality was essentially the same as example 1 (a colorless ester product of 40 APHA or less, no detectable odor by even a trained, human, perfume sampling expert, and a purity of at least 99.5 wt %).

### Example 3

Tetra-b-butyl titanate esterification catalyst was replaced by 2.5 grams zirconium carbonate (40% ZrO₂). The yield was 421 grams and the product quality was essentially the same as example 1.

### Example 4

Tetra-n-butyl titanate was replaced by 1.5 grams of methanesulfonic acid. When the esterification with n-hexanol was less than 4 mg KOH/g acidity, the reaction mass was cooled and neutralized with 10% aqueous sodium carbonate to a pH within the range of about 9-10. The aqueous phase was decanted, and the organic mass was vacuum steam stripped as in example 1 to remove excess n-hexanol until no n-hexanol remained in the ester product as determined by gas chromatography analysis.

The ester-containing reaction mass is cooled to 90° C., and 0.4 grams concentrated sulfuric acid is added dropwise with stirring. In drops, 166 grams of n-butyric anhydride is added with stirring at a rate sufficient to maintain a temperature between 90-110° C. The mass is stirred for 2 hours. Thereafter, by-product butyric acid is distilled off under full vacuum at 110° C.

The reaction mass is cooled to 70° C. and neutralized to pH of 10 with 10% aqueous sodium carbonate. The aqueous layer is decanted, and the organic ester layer is washed once with 250 ml water. The ester-containing product is then bleached and polished by adding 4 grams of 30% hydrogen peroxide and 4 grams of magnesium silicate by dropwise addition at a temperature of 70° C. The bleaching/polishing step is allowed to continue for 30 minutes at 70° C. whereupon water is removed by Karl Fischer titration using full vacuum to a water concentration of less than 0.1% by weight.

The bleached and polished acylated ester reaction mass is filtered to remove magnesium silicate. An acylated citrate triester is obtained in the amount of 480 grams that is colorless (40 APHA) and odorless with an assay of 99.6%.

### Example 5

The presence of a detectable odor was tested subjectively by a fragrance perfume chemist. A sample of n-butyryltri-n-hexyl citrate ester made by the process of the invention was compared with a commercially available product of the same citrate ester believed to be made according to U.S. Pat. No. 4,710,932. Samples of each citrate ester were warmed to 110° C. Test strips of a neutral material were then dipped in each sample and compared. The commercially available product exhibited a perceptible, acrid odor of butyric acid. The sample made according to the present invention had no discernible odor.

### Examples 6-8

Three samples of neat n-butyryltri-n-hexyl citrate (BTHC) were exposed to a temperature of 150° C. for 120 minutes, cooled to 25° C., and analyzed for volatile butyric acid odors using a same gas chromatograph-mass spectrometer. Example 6 included BTHC that was made in an esterification process that used an organic titanate catalyst and a bleaching/polishing process according to the invention. Example 7 used BTHC made with a methanesulfonic acid catalyst followed by neutralization, bleaching, and polishing steps as described herein. Example 8 used a commercially available BTHC sold under the name CITROFLEX B-6 by Morflex Chemical Company in Greensboro, N.C. which is believed to be made according to the process described in U.S. Pat. No. 4,710,532. Table 2 reports the results.

TABLE 2

| | | % Reduction in Odor |
|---|---|---|
| Example | Volatile Butyric Acid (ppm) | (relative to Ex. 8) |
| Ex. 6 | 0.6 | 96.7 |
| Ex. 7 | 1.0 | 96.1 |
| Ex. 8 | 25.8 | — |

### Examples 9-11

In examples 9-11, 100 phr medium molecular weight PVC, 50 phr of each plasticizer comprising n-butyryl-tri-n-hexyl citrate, 2.5 phr calcium/zinc stabilizer, and 0.25 phr stearic acid as lubricant were milled for 10 minutes at 325° F. (163° C.) and pressed into sheets that were 0.070 inches (1.78 mm) in thickness for 3 minutes at 32,000 psi. Samples were heated to 150° C. for two hours, cooled, and analyzed for butyric acid by gas chromatography-mass spectrometry (GC-MS).

Example 9 was n-butyryl-tri-n-hexyl citrate (BTHC) made with an organic titanate catalyst and treated with the bleaching/polishing process of the invention.

Example 10 was also n-butyryl-tri-n-hexyl citrate but made with methanesulfonic acid and the bleaching/polishing process of the invention.

Example 11 is the commercially available n-butyryl-tri-n-hexyl citrate believed to be made with an organic titanate catalyst according to U.S. Pat. No. 4,710,532 but without the bleaching/polishing process step of the invention.

| | | | Butyric Acid above |
|---|---|---|---|
| Example | Mfg Catalyst | Polishing? | Sample (ppm) |
| 9 | Titanate | Yes | 0.09 |
| 10 | methanesulfonic acid | Yes | 0.23 |
| 11 | Titanate | No | 2.11 |

The use of acylated ester plasticizer made according to the present process exhibits surprisingly significant reductions in volatile butyric acid relative to the commercially available BTHC, i.e., reductions in volatile butyric acid of at least 90% relative to BTHC made according to the process described in U.S. Pat. No. 4,710,532 in the absence of the present polishing step.

### Example 12

In example 12, n-hexanol was esterified with a methanesulfonic acid. Cyclohexane and cyclopentane were each useful as entraining agents for water removal as an azeotrope during the esterification reaction. Sufficient quantities of entraining agent were used to maintain the esterification reaction below about 110° C. The esterification product was then acylated with n-butyric anhydride and hypophosphorous acid as an acylation catalyst. The resulting BTHC was then treated with the bleaching/polishing step of the invention. The final product had a color of 25 APHA (essentially colorless) and was odor-free.

The examples presented herein are for the purpose of facilitating a description of the invention and are not intended to act as limitations on the scope of the appended claims. Each of the patents and disclosures cited herein is herein incorporated by reference.

1. A process for making a citrate ester that is free of human perceptible odor at 25° C. after heating for two hours at 150° C., said process comprising:
esterifying an alcohol with citric acid in the presence of an esterification catalyst under esterification conditions and removing excess alcohol to form an ester-containing reaction mixture,
acylating the ester-containing reaction mixture with an acid or acid anhydride under acylation conditions with an acylation catalyst for a time sufficient to form an acylated citrate ester,
removing by-product fatty acids and neutralizing the acylated citrate ester, and
polishing the acylated citrate ester with a polishing composition that comprises an absorbent for fatty acids.

2. A process according to claim 1 wherein said alcohol is an aliphatic alcohol having from 4 to 14 carbon atoms.

3. A process according to claim 1 wherein said alcohol is butanol, hexanol, or octanol.

4. A process according to claim 1 wherein said esterification catalyst is an organic titanate or an acid.

5. A process according to claim 4 wherein said esterification catalyst is tetra-n-butyl titanate.

6. A process according to claim 4 wherein said esterification catalyst is methanesulfonic acid.

7. A process according to claim 1 wherein acid or acid anhydride is acetic acid, acetic acid anhydride, butyric acid, or butyric acid anhydride.

8. A process according to claim 1 wherein acylation catalyst is sulfuric acid.

9. A process according to claim 1 wherein said absorbent is a finely divided solid.

10. A process according to claim 1 wherein said absorbent for fatty acid odors is an alkaline earth metal silicate.

11. A process according to claim 10 wherein said absorbent comprises magnesium silicate.

12. A polyvinyl chloride composition comprising a polyvinyl chloride resin and a plasticizing amount of n-butyryltri-n-hexyl citrate made according to the process of claim 1.

13. A polymeric composition comprising a polymeric resin and a plasticizing amount of n-butyryltri-n-hexyl citrate wherein said composition exhibits heat stability characteristics, after heating at 150° C. for two hours, of a color not greater than 60 APHA and no perceptible odor to humans at 25° C.

14. A composition according to claim 13 wherein said polymeric resin is polyvinyl chloride, polyvinylidene chloride, or polyvinylidene fluoride.

15. A composition according to claim 14 wherein said polymeric resin is polyvinyl chloride.

16. A medical article formed from the composition according to claim 13.

17. A medical article according to claim 16 in the form of a blood bag.

18. A medical article according to claim 17 wherein said polymeric resin is polyvinyl chloride.

19. A polymeric composition comprising a polymeric resin and a plasticizing amount of n-butyryltri-n-hexyl citrate made according to the process of claim 1.

20. Stable n-butyryltri-n-hexyl citrate made according to the process of claim 1.
